(19) United States
(12) Patent Application Publication
(10) Pub. No.: US 2016/0249866 A1
(43) Pub. Date: Sep. 1, 2016

(54) CRITERIA FOR VALID RANGE OF MOTION CAPTURE

(71) Applicant: Kitman Labs Limited, Dublin (IE)

(72) Inventor: Daniel Karl Ring, Dublin (IE)

(21) Appl. No.: 14/723,718

(22) Filed: May 28, 2015

Related U.S. Application Data
(60) Provisional application No. 62/121,934, filed on Feb. 27, 2015.

(51) Int. Cl. A61B 5/00; A61B 5/11

(52) U.S. Cl. CPC A61B 5/746; A61B 5/1128; A61B 5/1121; A61B 5/7282; A61B 5/742; A61B 5/4576; A61B 5/458; A61B 2503/10

(57) ABSTRACT

A range of motion is determined. Tracking information including a plurality of skeletal positions is received from an optical system. A plurality of vectors between selected ones of the skeletal positions where the plurality of vectors has an angular relationship is determined. It is determined whether the angular relationship meets a criteria. In the event the angular relationship does not meet the criteria, the angular relationship is rejected.

### CROSS REFERENCE TO OTHER APPLICATIONS

This application claims priority to U.S. Provisional Patent Application No. 62/121,934 entitled RANGE OF MOTION CAPTURE filed Feb. 27, 2015 which is incorporated herein by reference for all purposes.

### BACKGROUND OF THE INVENTION

In recent years, several attempts have been made to reduce the levels of athletic injury, from rule changes and better equipment to screening procedures and monitoring workloads. Despite this, the number of injuries in the majority of popular sports such as soccer, football, and baseball have either plateaued or continued to rise. Part of this injury trend may stem from the practical implications of screening an entire team of professional athletes consistently throughout a busy season. Although it is often desirable to screen athletes on a daily basis, the large amount of time required to prepare and consistently measure an athlete using traditional manual measurement tools is often difficult to reserve in an athlete's busy schedule. Therefore there exists a need for a more efficient and accurate way to screen an athlete.

### BRIEF DESCRIPTION OF THE DRAWINGS

Various embodiments of the invention are disclosed in the following detailed description and the accompanying drawings.

FIG. 1 is a block diagram illustrating an embodiment of a range of motion capture system.

FIG. 2 is a flow chart illustrating an embodiment of a process for capturing a range of motion of a subject.

FIG. 3 is a flow chart illustrating an embodiment of a process for processing optical data to determine a range of motion of a subject.

FIG. 4 is a flow chart illustrating an embodiment of a process for determining that a capture criteria has been met.

FIG. 5 is a flow chart illustrating an embodiment of a process for filtering measurements.

FIG. 6 is a flow chart illustrating an embodiment of a process for determining steadiness of a subject.

FIG. 7 is a flow chart illustrating an embodiment of a process for capturing a range of motion of a subject for a motion pattern.

FIG. 8 is a diagram illustrating an example embodiment of a user interface visual display of a range of motion capture.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Determining a measurement of a human subject is disclosed. For example, a range of motion of a joint of a human subject is measured. In some embodiments, tracking information includes a plurality of skeletal positions s received from an optical tracking system. For example, a camera image of an athlete has been taken and the image is processed to identify skeletal positions of the athlete that are received. In some embodiments, a 3-D camera is utilized to determine three-dimensional coordinates of skeletal positions. A plurality of vectors between selected ones of the skeletal positions having an angular relationship is determined. For example, a vector representing a limb segment of a human subject is determined and an angular relationship between selected vectors is determined. The angular relationship is tracked over time, whereby a sequence of relationships is detected. From the sequence of relationships, the range of motion is determined over time. For example, the angular relationship represents a range of motion angle of a joint of an athlete and the angle is tracked over time to detect any significant changes that may indicate an increased risk of injury.

In some embodiments, when the angular relationship is detected, it is determined whether the angular relationship meets a criteria. For example, not all determined angular relationships may be suitable for determining the correct range of motion because the subject may be in an incorrect position and/or in the middle of demonstrating the range of motion. In some embodiments, only when it is detected that the subject is in the correct and steady final position can a detected angular relationship be utilized to determine the range of motion. In the event the angular relationship does not meet the criteria, the tracking information is rejected. For example, the detected angular relationship of the tracking information is rejected and not utilized to determine the range of motion.

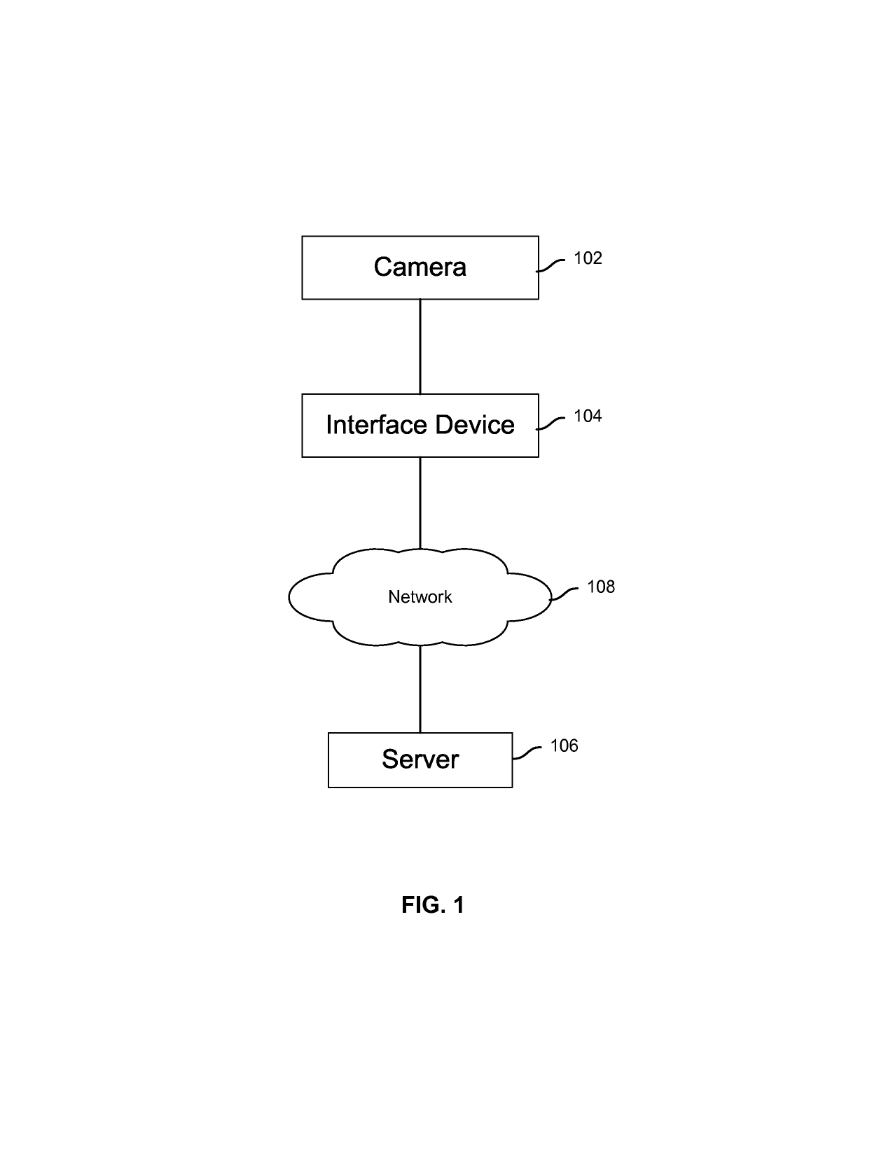

FIG. 1 is a block diagram illustrating an embodiment of a range of motion capture system. Camera 102 is connected to user device 104. For example, camera 102 is utilized to capture images of an athlete subject to automatically measure and determine one or more range of motion measurements for the athlete. Camera 102 may be a webcam, a video camera, a 2-D image camera, a 3-D image camera, a stereo camera, a sheet of light triangulation camera, a structured light camera, a time-of-flight camera, an interferometry camera, a coded aperture camera, a light field camera, an infrared camera, and/or any other imaging or optical device. In some embodiments, an image/video captured by camera 102 is analyzed to identify skeletal positions (e.g., points on a body) of a human subject captured by camera 102. Examples of user device 104 include a tablet computer, a laptop computer, a desktop computer, a wearable computer, a smartphone, and/or any other type of computing device. In some embodiments, user device 104 is a special purpose proprietary device/computer for tracking range of motion data. In some embodiments, camera 102 is integrated together with user device 104 in a single device. For example, a tablet computer includes an integrated three-dimensional camera.

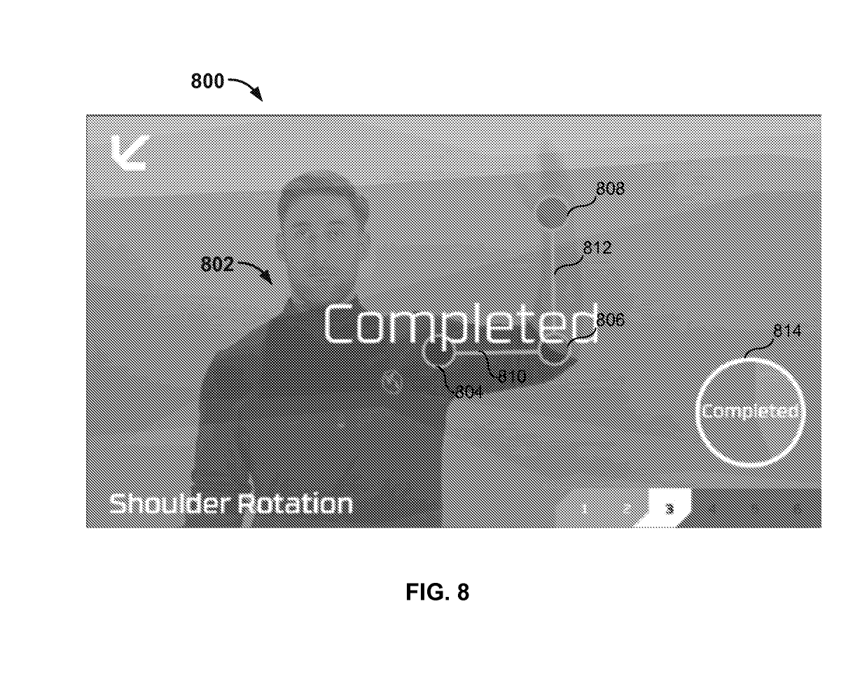

In some embodiments, user device 104 includes a display that provides visual feedback about skeletal positions detected and/or actions to be performed by the subject. An example of the visual feedback is shown in FIG. 8. In some embodiments, a user utilizes user device 104 to provide information about the user. For example, information such as weight, height, diet, muscle soreness, injury, mood, sleep quality/quantity, training metrics, stress level, fatigue level, etc. may be provided via user device 104 for tracking in a user profile. In some embodiments, a user utilizes user device 104 to log in to a user profile/account of the user. The user profile/account of the user may track information about the user including a history of range of motion data. User device 104 is connected to server 106 via network 108. The user profile/account of users may be managed and/or stored by server 106. For example, server 106 provides network cloud storage and processing capabilities.

In some embodiments, server 106 and/or user device 104 processes information about a subject, including a history of range of motion data, to automatically determine a likelihood of injury of the subject. For example, anomalies in range of motion are detected and correlated with data on risk of injury associated with the anomalies. In some embodiments, based on the analysis, a warning, a message, a suggested exercise, a medical recommendation, an advice, and/or any other related information may be provided to a user (e.g., via user device 104).

The shown connections between the components of FIG. 1 may be a wired and/or wireless connection. One or more of the following may be included in network 108: a direct or indirect physical communication connection, mobile communication network, Internet, intranet, Local Area Network, Wide Area Network, Storage Area Network, a wireless network, a cellular network, and any other form of connecting two or more systems, components, or storage devices together. Additional instances of any of the components shown in FIG. 1 may exist. For example, multiple cameras may be connected to the user device and may be utilized to capture range of motion for a single subject. Server 106 may be one of a plurality of distributed servers that process network connected device data. In some embodiments, components not shown in FIG. 1 may also exist.

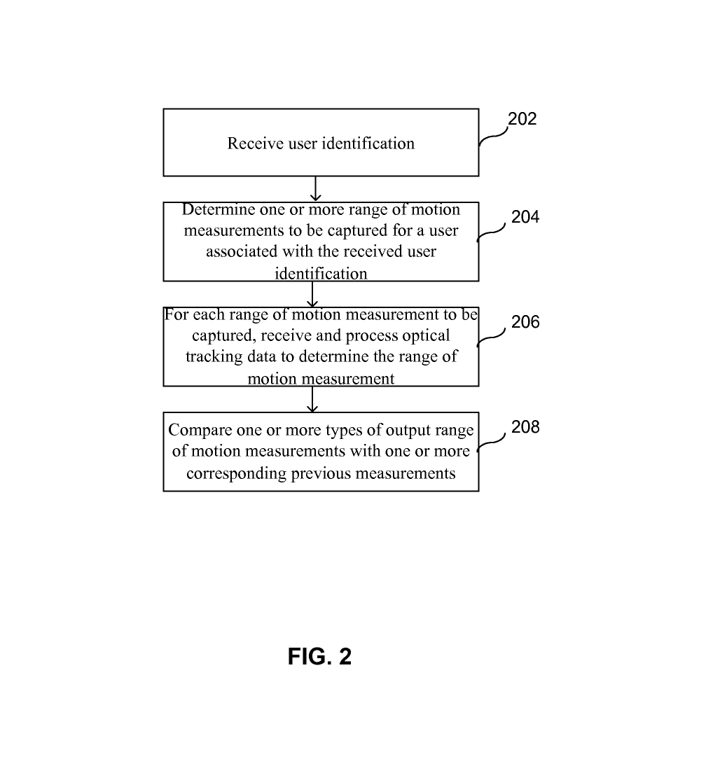

FIG. 2 is a flow chart illustrating an embodiment of a process for capturing a range of motion of a subject. In some embodiments, at least a portion of the process of FIG. 2 may be performed by camera 102, user device 104 and/or server 106 of FIG. 1.

At 202, user identification is received. In some embodiments, receiving the user identification includes receiving a username and/or password of a user subject to be analyzed. In some embodiments, the user identification is automatically determined. For example, biometric information about a user is automatically detected and the biometric information is utilized to verify the identity of the user and/or identify a user account/profile of the user. Examples of the biometric information include a finger print, facial recognition information, ear shape information, body proportion information, skeletal position/proportion/geometry information, eye (e.g., iris, retina, etc.) information, vein recognition, movement signature recognition, voice recognition, and any other type of biometric information. The biometric information may be obtained using an optical sensor such as camera 102 of FIG. 1. In some embodiments, a profile of a user identified by the user identification is obtained by a user device from a server via a network. In some embodiments, the user identification is associated with a manager of one or more user subjects to be profiled. For example, a trainer for an athletic team signs into an athletic profiler system to start the profiling of each team member.

At 204, one or more range of motion measurements to be captured for a user associated with the received user identification are determined. For example, the each type of measurement is to be measured based on its associated measurement schedule and the types of measurements due for each measurement session are determined. In some embodiments, the types of range of motion measurements to be captured are dynamically based on a profile of a user subject to be measured. For example, the type/amount of sports, activity, workout, etc. performed and/or to be performed may be utilized to dynamically determine types of range of motion measurements to be captured. Examples of other factors utilized to determine the range of motion measurements to be captured include injury information (e.g., an injury may prevent a pose required for measurement, measurements related to previous injuries are to be obtained, etc.), medical information, diet, muscle soreness, mood, sleep quality/quantity, training metrics, stress level, fatigue level, etc. In some embodiments, the range of motion measurement includes an angular measurement. In some embodiments, the range of motion measurement includes an angular relationship between two body parts. For example, an angular relationship between body parts connected at a joint is the range of motion measurement.

At 206, for each range of motion measurement to be captured, optical tracking data is received and processed to determine the range of motion measurement. In some embodiments, in order to measure a range of motion, a user subject is required to demonstrate a pose that demonstrates a range of motion in front of an optical tracking device. For example, a display of a user device provides an instruction on the pose to be demonstrated and the user subject is able to view on the screen the image of the user being captured by a camera optical tracking device. If the user subject requires assistance in performing the pose, the user may be provided video coaching assistance. For example, a user may indicate to view a video demonstrating the pose to be performed. In some embodiments, based on a detection position of one or more points on a body of the user subject, a specific message/coaching/advice on how to correct the current pose of the user (e.g., specific portion of the pose) to perform the desired pose is provided. In some embodiments, the optical data is obtained from camera 102 of FIG. 1. In some embodiments, updated optical data is continually received. For example, as updated images of a user subject are captured and processed, updated sets of skeletal position coordinates at different time instances are received. Examples of the optical data include an image, a video, coordinates, and other body position data.

In some embodiments, determining the range of motion measurement includes detecting that the user subject is in the correct position for the specific type of range of motion measurement to be captured. For example, skeletal position coordinates are analyzed to determine whether the user subject is in the correct pose/position. The correct position may be determined by identifying that one or more relationships between vectors defined by skeletal position coordinates and/or between skeletal position coordinates are within a threshold. In some embodiments, an indication of correctness of a pose being demonstrated by the user subject is provided (e.g., see FIG. 8). For example, a visual color coded indication (e.g., red for incorrect pose, yellow for pose that is close to the acceptable pose, and green for acceptable pose) is displayed to the user. In some embodiments, a numerical indication of how close the detected pose of the user is to the ideal pose for the type of range of motion measurement to be obtained is provided. For example, a numerical indication that is a measure of the idealness of the pose is provided to encourage the user to provide the best pose for each measurement.

In some embodiments, in order to improve the consistency of the subject's pose for the desired range of motion measurement, the user subject is requested to steadily hold a pose demonstrating the range of motion for at least a set amount of time. The range of motion measurement may be determined using optical data received while the user subject is detected as steady in the correct pose for the measurement. In some embodiments, a measure of steadiness is determined and the user is determined to be steady when the steadiness measure is within a threshold. In some embodiments, the range of motion measurement is determined only using skeletal position data detected to be stable. In some embodiments, the range of motion measurement is determined by averaging a plurality of measurements detected while the user is detected as being steady.

Examples of the poses for the different types of range of motion measurement include external shoulder rotation for left and right arms, internal shoulder rotation for left and right arms, ankle dorsiflexion for left and right ankles, hip rotation (in flexion) for left and right hips, and hamstring sit and reach. In some embodiments, the range of motion measurement to be captured is detected from a gesture/motion/action. For example, a user performs an activity in front of a camera and one or more range of motion measurements are determined from the captured series of movements of the user subject. An example of the activity includes a baseball pitch using the left/right arm and the range of motion of the shoulder while performing the baseball pitch is determined.

At 208, one or more types of output range of motion measurements are compared with one or more corresponding previous measurements. In some embodiments, the comparison is utilized automatically to determine a likelihood of injury of the subject. For example, anomalies in range of motion measurements over time are detected and correlated with data on risk of injury associated with the anomalies. In some embodiments, based on the analysis, a warning, a message, a suggested exercise, a medical recommendation, an advice, and/or any other information may be provided to a user. In some embodiments, one or more of the following types of data about the user subject may be analyzed together with the range of motion measurements: weight, height, diet, muscle soreness, prior/current injury, mood, sleep quality/quantity, training metrics, athletic performance, stress level, fatigue level, etc. In some embodiments, a historical average, median, mode, maximum, minimum, and any other statistic of the range of motion over time are determined. For example, a current range of motion measurement is compared with a historical average of the range of motion measurement, and in the event the difference is greater than a threshold value, a message (e.g., injury risk warning) is provided.

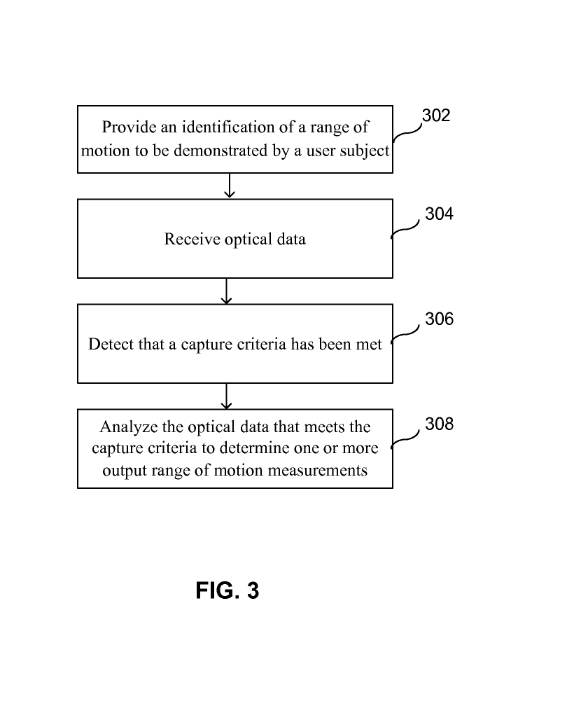

FIG. 3 is a flow chart illustrating an embodiment of a process for processing optical data to determine a range of motion of a subject. In some embodiments, at least a portion of the process of FIG. 3 may be performed by camera 102, user device 104 and/or server 106 of FIG. 1. In some embodiments, the process of FIG. 3 is included in 206 of FIG. 2.

At 302, an identification of a range of motion to be demonstrated by a user subject is provided. For example, an identification of one of the range of motion measurements identified in 204 of FIG. 2 is displayed on a display of user device 104 of FIG. 1. In some embodiments, the user subject is prompted to be positioned in front of a camera such that the entire body of the subject is captured by the camera. For example, a display displays an image captured by a camera and the user may modify the position of the user and/or the camera to ensure that the entire body of the subject is being captured by the camera. In some embodiments, to measure a range of motion, a user subject is required to demonstrate a pose that demonstrates the range of motion. If the user subject requires assistance in performing the pose, the user may be provided video coaching assistance. For example, a user may indicate to view a video demonstrating the pose to be performed.

At 304, optical data is received. In some embodiments, the optical data includes data captured using a camera (e.g., camera 102 of FIG. 1). In some embodiments, receiving the optical data includes periodically receiving a set of data corresponding to skeletal positions of a user at an updated point in time. For example, each set of optical data includes data captured at a specific moment in time and as new data is detected by a camera, the new data (e.g., identifies a current position/pose of the user subject) is received from the camera. In some embodiments, the optical data includes coordinates of skeletal positions on the user subject's body. For example, coordinates (2-D or 3-D) of one or more of the following skeletal positions are provided: right hand, right wrist, right elbow, right shoulder, head, shoulder center, left hand, left wrist, left elbow, left shoulder, spine, hip center, right hip, right knee, right ankle, right foot, left hip, left knee, left ankle, and left foot. In some embodiments, the optical data is received via a software development kit (i.e., SDK) and/or an application programming interface (i.e., API) of a camera. For example, a 3-D camera (e.g., Microsoft Kinect) captures an image of a subject and processes the image using a processor of the camera to identify 3-D coordinates of skeletal positions on the body of the subject. These 3-D coordinates may be accessed from the 3-D camera using a SDK/API of the camera (e.g., accessed by user device 104 from camera 102 of FIG. 1). The set of detected skeletal positions of the subject may be updated/received at periodic intervals to track the updated movement of the subject.

At 306, it is detected that a capture criteria has been met. In some embodiments, the capture criteria is met when it is at least detected that the user subject is in the correct position for the specific type of range of motion measurement to be captured. For example, skeletal position coordinates are analyzed to determine whether the user subject is in the correct pose. The correct position may be identified by determining that the one or more relationships between vectors defined by skeletal position coordinates and/or between skeletal position coordinates are within a threshold. In some embodiments, an indication of correctness of the pose being performed by the user subject is provided (e.g., see FIG. 8). For example, a visual color coded indication (e.g., red for incorrect pose, yellow for pose that is close to the acceptable pose, and green for acceptable pose) is displayed to the user. In some embodiments, a numerical indication of how close the detected pose of the user is to the ideal pose for the type of measurement to be obtained is provided. For example, the correctness of the measurement may be highly dependent on the user subject providing a correct and consistent pose, and a numerical indication that is a measure of the idealness of the pose is provided to encourage the user to provide the best pose for each measurement. In some embodiments, based on a detected position of one or more points on a body of the user subject, a specific message/coaching/advice on how to correct the current pose of the subject (e.g., indication on which pose component is incorrect and suggestion how to correct) to achieve the desired pose is provided.

In some embodiments, in order to improve the consistency of the pose for the desired range of motion measurement, the user subject is required to steadily hold a pose for at least a set amount of time. For example, the capture criteria is met when it is at least detected that the subject has held the correct pose for at least a first threshold amount of time. The user may be required to hold the pose for at least an additional second threshold amount of time while the range of motion measurement is determined. In some embodiments, a measurement of steadiness is determined and the user is determined to be steady when the steadiness measurement is within a steadiness threshold.

In some embodiments, the optical data is filtered. For example, optical data is filtered to smooth out data outliers that may be caused by noise. The noise may be caused by optical sensor noise, optical capture environment noise, a resolution limit, a data modeling artifact, and/or any other type of noise that may cause erratic undesired variation in optical data not directly attributable to movement of the user subject. In some embodiments, filtering the optical data includes removing outlier data. For example, based on a known Gaussian distribution for the data to be filtered, outlier data is discarded.

At 308, the optical data that meets the capture criteria is analyzed to determine one or more output range of motion measurements. In some embodiments, the range of motion measurements include an angular measurement and/or a length measurement. For example, the angular relationship between body parts connected at a joint is measured as the range of motion measurement. In another example, a distance between body parts while a user subject is performing a stretch pose is measured as the range of motion measurement. In some embodiments, the range of motion measurement is determined only using skeletal position data detected to be stable and in the correct position. In some embodiments, for a specific type of range of motion measurement to be determined, the range of motion measurement samples are determined and averaged while the pose of the subject is determined to be stable and correct. For example, after it is detected that a subject user is in the correct stable pose for one second, a sample range of motion measurement is determined for each updated set of skeletal position data (e.g., received every millisecond) belonging to the next two seconds while the subject is holding the stable and correct pose. The range of motion measurements belonging to these two seconds may be averaged to determine an output range of motion measurement. In some embodiments, rather than utilizing the average value, a median or mode value is set as the output range of motion value.

In some embodiments, determined sample range of motion measurements are filtered. For example, sample range of motion measurements that are utilized to determine the output range of motion measurement do not include measurements that have been filtered out to smooth out data outliers caused by noise. The noise may be caused by optical sensor noise, optical capture environment noise, a resolution limit, a data modeling artifact, and/or any other type of noise that may cause erratic undesired variation in optical data not directly attributable to movement of the user subject. In some embodiments, based on a known Gaussian distribution for the range of motion measurement, outlier data is discarded. In some embodiments, the output range of motion measurement is utilized in 208 of FIG. 2 and compared with one or more corresponding previous output measurements.

In one example, for the shoulder rotation (for either external or internal and either left or right), its range of motion measurement is determined by projecting a three-dimensional elbow-to-wrist vector, V_{ew}=P_{w}−Pₑ, onto a plane perpendicular to a shoulder-to-elbow vector, Vₛₑ=Pₛ−Pₑ, and taking the two-dimensional angle difference between the projected vector and the projected “left” vector, V_{left}=(1, 0, 0). P_{w} is the three-dimensional skeletal wrist position coordinate, Pₑ is the three-dimensional skeletal elbow position coordinate and Pₛ is the three-dimensional skeletal shoulder position coordinate of the body side (e.g., left or right) of interest. In other words, the shoulder range of motion measurement is angle θ=Angle of projection(R*V_{ew})−Angle of projection(V_{left}), where R is the Quaternion rotation matrix with rotation given by V_{left}×Vₛₑ and normalized appropriately. One or more shoulder rotation range of motion measurement samples may be averaged to determine the output shoulder rotation range of motion measurement.

In another example, for the ankle dorsiflexion (for either left or right), its range of motion measurement is determined by determining the difference in angle between a down vector V_{down}=(0, −1, 0) and an ankle-to-knee vector Vₐₖ=Pₖ−Pₐ·Pₖ is the skeletal knee position coordinate and Pₐ is the skeletal ankle position coordinate, both on the body side (e.g., left or right) of interest. In other words, ankle dorsiflexion range of motion measurement is angle θ=angle between V_{down} and Vₐₖ. One or more ankle dorsiflexion range of motion measurement samples may be averaged to determine the output ankle dorsiflexion range of motion measurement.

In another example, for hip rotation, in flexion, (for either left or right) its range of motion measurement is determined by projecting a three-dimensional knee-to-ankle vector, Vₖₐ=Pₐ−Pₖ, onto a plane perpendicular to a hip-to-knee vector, Vₕₖ=Pₖ to Pₕ and measuring the angle between the projected vector and the projected “left” vector, V_{left}=(1, 0, 0). Pₐ is the three-dimensional skeletal ankle position coordinate, Pₖ is the three-dimensional skeletal knee position coordinate and Pₕ is the three-dimensional skeletal hip position coordinate, all of the body side (e.g., left or right) of interest. In other words, the hip rotation range of motion measurement is angle θ=Angle of projection(R*Vₖₐ)−Angle of projection(V_{left}), where R is the Quaternion rotation matrix with rotation given by V_{left}×Vₕₖ and normalized appropriately. One or more hip rotation range of motion measurement samples may be averaged to determine the output hip rotation range of motion measurement.

In another example, for hamstring sit and reach, its range of motion measurement is determined by measuring the difference in depth/distance between the subject's hands and the subject's feet. For example, hamstring sit and reach range of motion measurement distance equals the distance between a skeletal foot position coordinate and a skeletal hand position coordinate. One or more hamstring sit and reach range of motion distance measurement samples may be averaged to determine the output hamstring sit and reach range of motion distance measurement.

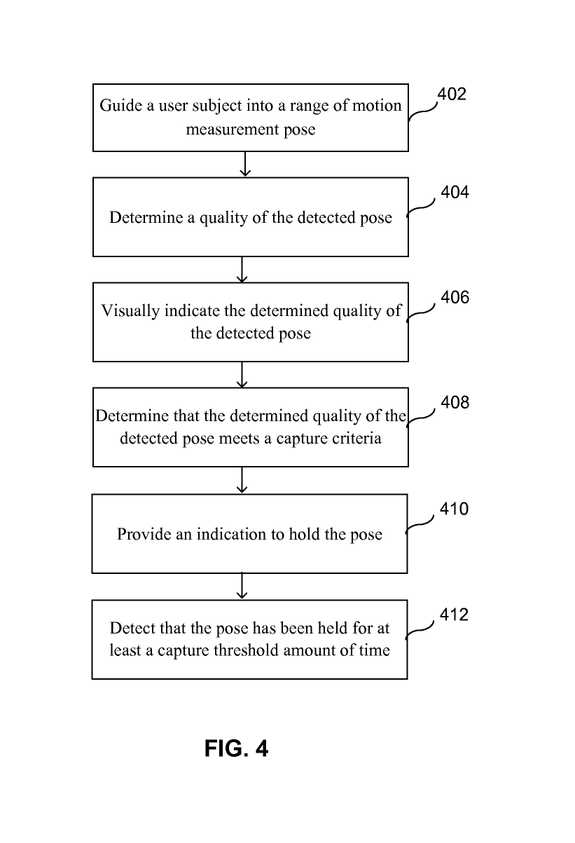

FIG. 4 is a flow chart illustrating an embodiment of a process for determining that a capture criteria has been met. In some embodiments, at least a portion of the process of FIG. 4 may be performed by user device 104 and/or server 106 of FIG. 1. In some embodiments, the process of FIG. 4 is included in 206 of FIG. 2. In some embodiments, the process of FIG. 4 is included in 306 of FIG. 3.

At 402, a user is guided into a range of motion measurement pose. For example, an identification of one of the range of motion measurements identified in 204 of FIG. 2 is displayed on a display of user device 104 of FIG. 1. In some embodiments, instructions on the pose to be performed by a subject are provided. If the user subject requires assistance in performing the pose, the user may be provided video coaching assistance. For example, a user may indicate to view a video demonstrating the pose to be performed. In some embodiments, a current skeletal position of the subject is detected and dynamic guidance is provided on how to change the current detected pose of the subject to the correct pose of the range of motion measurement to be captured.

An example type of range of motion measurement is an exterior shoulder rotation range of motion measurement either for the left shoulder or right shoulder. In order to guide a subject into the pose for this type of range of motion measurement, the subject may be instructed to stand in front of an optical capture device, raise the appropriate arm (e.g., left or right depending on whether left or right shoulder rotation is being measured) such that the subject's elbow is in line with their shoulders and bend the elbow to create a 90 degree relationship between the upper arm and the lower arm at the elbow, and rotate the appropriate shoulder backwards as far as possible.

Another example type of range of motion measurement is an interior shoulder rotation range of motion measurement either for the left shoulder or right shoulder. In order to guide a subject into the pose for this type of range of motion measurement, the subject may be instructed to stand in front of an optical capture device, raise the appropriate arm (e.g., left or right depending on whether left or right shoulder rotation is being measured) such that the subject's elbow is in line with their shoulders and bend the elbow to create a 90 degree relationship between the upper arm and the lower arm at the elbow, and rotate the appropriate shoulder forwards as far as possible.

Another example type of range of motion measurement is an ankle dorsiflexion range of motion measurement either for the left shoulder or right ankle. In order to guide a subject into the pose for this type of range of motion measurement, the subject may be instructed to stand in front of an optical capture device, extend the appropriate ankle (left or right ankle) forward in front, place the subject's hands on the subject's waist, and while keeping the back straight, bend the appropriate ankle to its extreme while keeping the heel on the ground.

Another example type of range of motion measurement is a hip rotation, in flexion, range of motion measurement either for the left shoulder or right hip. In order to guide a subject into the pose for this type of range of motion measurement, the subject may be instructed to sit on a chair in front of an optical capture device, bend the knee on the side of the hip to be measured approximately 90 degrees, and rotate the appropriate hip (left or right) by swinging the ankle on the appropriate side outwards as far as possible.

Another example type of range of motion measurement is a hamstring stretch range of motion measurement. In order to guide a subject into the pose for this type of range of motion measurement, the subject may be instructed to sit on a flat surface in front of an optical capture device with legs extended straight out and closed together, and reach forward with the hands towards the toes while keeping at least a set minimum distance above the feet.

At 404, a quality of the detected pose is determined. For example, optical data is received and processed to determine an indication of correctness of the pose being performed by the user subject. In some embodiments, for each type of range of motion measurement, there exists one or more quality measurement factors that identify the correctness of the pose for the specific range of motion measurement. For example, each quality measurement is determined using a quality measurement formula that measures how close a detected pose of the user is to the ideal pose for the specific type of range of motion measurement to be obtained. In some embodiments, each quality measurement is associated with a correctness threshold and/or a correctness range. For example, in order to determine that a pose of a subject is correct and meets a capture criteria, one or more quality factors associated with the range of measurement must meet an appropriate threshold/range. In some embodiments, an overall quality measurement for a range of motion measurement is determined using one or more component quality measurements for the pose of the range of motion measurement. For example, a plurality of component quality measurements are multiplied together to determine a single overall quality measurement for the range of motion measurement. In some embodiments, determining the quality measurement includes determining one or more angular relationships between body parts of the user that are different from the one or more angular relationships for the range of motion measurement. In some embodiments, determining the quality of the detected pose includes determining whether the captured subject is at a sufficient distance from an optical sensor and/or the appropriate body parts of the subject have been captured by an optical sensor.

In one example, for the shoulder rotation (for either external or internal and either left or right) range of motion measurement, two quality measurements are determined to determine the quality of the detected pose for the shoulder rotation range of motion measurement. The first quality measurement is the shoulder correctness measurement that identifies how much a user subject is keeping their elbow in line with their shoulder. An example formula of this quality measurement is given by T₁=max(0, min(1, 1−(θ−13°)/50°)), where θ is the angle between a shoulder vector (e.g., vector defined from one shoulder skeletal coordinate to the other shoulder skeletal coordinate) and an upper arm vector (e.g., vector defined from left shoulder skeletal coordinate to the left elbow skeletal coordinate). In some embodiments, T₁ must be greater than 0.7 to determine that a correct shoulder position for the shoulder rotation range of motion measurement pose has been detected. The second quality measurement is the elbow correctness measurement that identifies whether a subject has been sufficiently bent. An example formula of this quality measurement is given by T₂=max(0, min(1, θ/80°)), where θ is the angle between an elbow-to-wrist vector (e.g., vector defined from left elbow skeletal coordinate to the left wrist skeletal coordinate) and an upper arm vector (e.g., vector defined from left shoulder skeletal coordinate to the left elbow skeletal coordinate). In some embodiments, T₂ must be greater than 0.7 to determine that an arm has been sufficiently bent for the shoulder rotation range of motion measurement. In some embodiments, an overall quality measurement for a specific shoulder rotation range of motion measurement is determined by multiplying together T₁ and T₂.

In another example, for the ankle dorsiflexion (for either left or right) range of motion measurement, quality of the detected pose is determined by determining whether one or more pose criteria have been satisfied. For example, in order to detect that a correct pose to measure the ankle dorsiflexion range of motion measurement has been demonstrated, it is determined whether a subject's heel is touching the ground and whether the appropriate knee has been bent to at least a minimum angle (e.g., 15 degrees). The detection of the pose criteria may be performed using one or more quality measurement formulas (e.g., using one or more skeletal point coordinates of the optical data) and/or machine learning (e.g., gestures learning).

In another example, for the hip rotation (for either left or right) range of motion measurement, quality of the detected pose is determined by determining whether one or more pose criteria have been satisfied in addition to determining a quality measurement. An example pose criteria for the hip rotation range of motion measurement includes detecting whether a user subject is sitting on a chair. The detection of this pose criteria may be performed using one or more quality measurement formulas (e.g., using one or more skeletal position coordinates of the optical data) and/or machine learning (e.g., gestures learning). Another example of the pose criteria includes detecting whether a user has rotated the appropriate hip to at least a minimum angle (e.g., 15 degrees). In some embodiments, a bent knee quality measurement is determined for the hip rotation range of motion measurement. This bent knee quality measurement identifies that the appropriate knee has been sufficiently bent. An example formula for the bent knee quality measurement is given by T_{Bent Knee}=1−max(0, min(1, 1−(|θ−90°|−15°)/(30°−15°))) where θ is the angle between a knee-to-ankle vector (e.g., vector defined from left knee skeletal coordinate to the left ankle skeletal coordinate) and a hip-to-knee vector (e.g., vector defined from left hip skeletal coordinate to the left knee skeletal coordinate). In some embodiments, T_{Bent Knee} must be greater than 0.5 to determine that a correct bent knee angle for the hip rotation range of motion measurement pose has been detected.

In another example, for the hamstring sit and reach (for either left or right) range of motion measurement, quality of the detected pose is determined by determining whether one or more pose criteria have been satisfied. For example, in order to detect that a correct pose to measure the hamstring sit and reach range of motion measurement has been demonstrated, it is determined whether a subject is sitting down on the ground and whether the hand of the subject is sufficiently higher off the ground (e.g., minimum vertical distance value) as compared to the feet of the subject. The detection of the pose criteria may be performed using one or more quality measurement formulas (e.g., using one or more skeletal point coordinates of the optical data) and/or machine learning (e.g., gestures learning).

At 406, the determined quality of the detected pose is visually indicated. An example is shown in FIG. 8. In some embodiments, a visual color coded indication (e.g., red for incorrect pose, yellow for pose that is close to the acceptable pose, and green for acceptable pose) is displayed to the user. For example, an image of a wire skeleton of detected skeletal points of a subject is displayed and colored in different colors to indicate the quality of the detected pose. In some embodiments, each visual color corresponds to a different specified predetermined range for one or more quality measurement values. For example, an overall quality measurement indicator (e.g., numeric indicator) is determined for a current detected pose of the subject and the visual color corresponding to the value range that includes the value of the indictor is selected as the visual color coded indication. The value ranges corresponding to each color may vary depending on the type of pose/measurement. In some embodiments, the value ranges corresponding to colors may be associated with the number of quality conditions satisfied. For example, red is selected for zero conditions satisfied, yellow is for one to two conditions satisfied, and green is for all three conditions satisfied. In some embodiments, the value ranges may be associated with both the number of conditions satisfied and numerical value range of a quality measurement value.

In some embodiments, one or more numerical quality scores indicating the determined quality are provided. For example, although the quality of the detected pose is sufficient to determine a range of motion measurement, the detected pose could be improved and a quality score is provided to encourage the subject to increase the quality score. In some embodiments, the quality score is associated with an overall quality measurement determined in 404. In some embodiments, the quality score is associated with one or more quality measurement formulas specific to a particular type of range of motion measurement to be determined. In some embodiments, the quality score is associated with a number of conditions satisfied. For example, a fixed score amount is added to the quality score in the event a quality condition is satisfied (e.g., a component score for detecting that the user is sitting on a chair for ankle dorsiflexion range of motion measurement). In some embodiments, the quality score associated with a range of motion measurement is stored along with the range of motion measurement to indicate the quality of the measurement. In some embodiments, the quality score is a numerical indication of how close the detected pose of the user is to the ideal pose for the type of measurement to be obtained. For example, the correctness of the range of motion measurement may be highly dependent on the user subject providing a correct and consistent pose for the range of motion measurement, and a numerical indication that is a measure of the idealness of the pose is provided to encourage the user to provide the best pose for each measurement.

In some embodiments, a subject is provided the quality score and not provided the exact range of motion measurement. For example, providing the exact range of motion measurement to a competitive athlete might encourage the athlete to attempt for the largest range of motion measurement possible at the expense of poor pose form. However, detection of metrics such as injury risk may largely depend on detecting variations and changes in measurements over time that are measured from consistent and ideal pose forms. Thus to discourage competition for the largest/best range of motion measurement value and encourage best and consistent pose quality, subject may be provided the quality score and not provided the exact range of motion measurement to encourage increases in the quality score rather than the range of motion measurement value.

At 408 it is determined that the determined quality of the detected pose meets a capture criteria. In some embodiments, the capture criteria is specific to the specific type of pose and/or type of range of motion being determined. In some embodiments, determining that the quality of the detected pose meets the capture criteria includes determining that determined quality meets a threshold associated with the capture criteria. For example, it is determined that all component quality measurements and/or conditions meet a respective threshold. In another example, it is determined that an overall quality measurement meets a threshold. In some embodiments, it is determined that the determined quality of the detected pose meets the capture criteria in the event the determined quality meets a threshold for at least a threshold amount of time.

At 410, an indication to hold the pose is provided. For example, a visual instruction is provided to indicate to a user subject to hold the current pose of the user detected to be sufficient to determine the range of motion measurement while the range of motion measurement is being determined. In some embodiments, the indication to hold the pose is only provided after it is determined that the determined quality of the detected pose meets the capture criteria and continually/periodically detected range of motion measurements have been steady for at least a preliminary threshold amount of time. In some embodiments, a countdown of the remaining time to hold the pose steady is indicated to the user. In some embodiments, if the subject does not consistently hold the pose for at least a threshold amount of time, the process returns to 408 and the pose must be held again for at least the threshold amount of time from the beginning

At 412, it is detected that the pose has been held for at least a capture threshold amount of time. In some embodiments, detecting that the pose has been held for at least the capture threshold time includes determining that a continually/periodically determined quality of the detected pose (e.g., filtered data) has met the capture criteria for at least the capture threshold amount of time and that continually/periodically detected range of motion measurement samples (e.g., filtered data) have been steady for at least the capture threshold amount of time. For example, as new/updated optical data including skeletal point information is continually received, each set of data is processed to determine the quality measurement and the range of motion measurement sample associated with the set of data. The optical data, quality measurement, and/or the range of motion measurement sample of the set of data may be filtered to reject any anomalous data that is not to be utilized to detect pose quality, pose steadiness, and/or range of motion measurement. The filtering may be utilized to reject spurious data caused by optical sensor noise. In some embodiments, the continually/periodically detected range of motion measurements are stable in the event differences between the valid detected range of motion measurements are within a predetermined range. In some embodiments, at least a portion of the range of motion measurement samples associated with the held pose is utilized to determine an output range of motion measurement. For example, a plurality of range of motion measurement samples associated with the held pose is averaged. The portion of the range of motion measurement samples utilized to determine the output range of motion measurement may only include the range of motion measurement samples determined within a portion of the capture threshold amount of time.

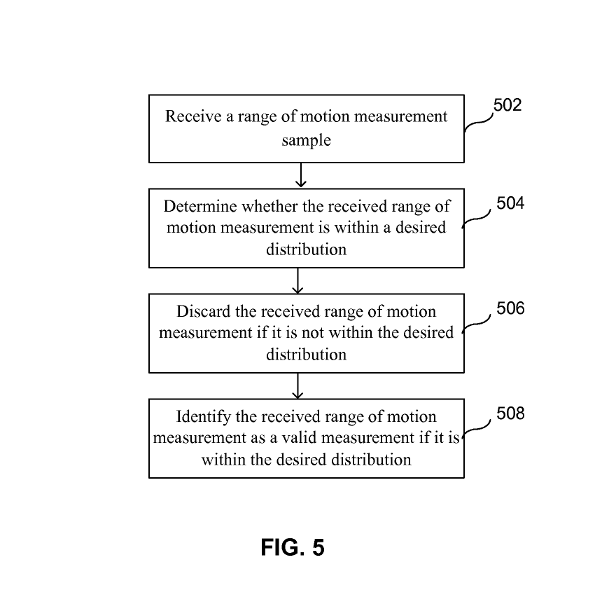

FIG. 5 is a flow chart illustrating an embodiment of a process for filtering measurements. In some embodiments, at least a portion of the process of FIG. 5 may be performed by camera 102, user device 104 and/or server 106 of FIG. 1. In some embodiments, the process of FIG. 5 is included in 206 of FIG. 2. In some embodiments, the process of FIG. 5 is included in 306 and/or 308 of FIG. 3. In some embodiments, the process of FIG. 5 is included in 408, 410 and/or 412 of FIG. 4. For example, optical data is filtered to smooth out data outliers that may be caused by noise. The noise may be caused by optical sensor noise, optical capture environment noise, a resolution limit, a data modeling artifact, and/or any other type of noise that may cause erratic undesired variation in optical data not directly attributable to movement of the user subject. In some embodiments, filtering measurements include identifying and removing outliers and smoothing and resampling measurement data.

At 502, a range of motion measurement sample is received. In some embodiments, the received range of motion measurement sample has been determined using optical data received in 206 of FIG. 2. For example, an updated set of optical data is continually received on a periodic basis and each latest set of optical data (e.g., including coordinates of a complete skeletal pose detected at a single moment) is analyzed to determine the range of motion measurement sample for the set. In some embodiments, the range of motion measurement sample is stored in a data structure. For example, the received range of motion measurement sample is stored in a first-in-first-out fixed size data structure (e.g., circular list, where for each range of motion measurement sample added past a fixed size limit, the earliest sample entry is removed to ensure only the fixed size limit number of range of motion measurements is stored). In some embodiments, the size of the data structure is at least large enough to store all range of motion measurement samples of optical data captured during the capture threshold amount of time of 412 of FIG. 4.

In one example, for the shoulder rotation range of motion measurement (for either external or internal and either left or right), its range of motion measurement has been determined by projecting a three-dimensional elbow-to-wrist vector, V_{ew}=P_{w}−Pₑ, onto a plane perpendicular to a shoulder-to-elbow vector, Vₛₑ=Pₛ−Pₑ, and taking the two-dimensional angle difference between the projected vector and the projected “left” vector, V_{left}=(1, 0, 0). P_{w} is the three-dimensional skeletal wrist position coordinate, Pₑ is the three-dimensional skeletal elbow position coordinate and Pₛ is the three-dimensional skeletal shoulder position coordinate of the body side (e.g., left or right) of interest. In other words, the shoulder range of motion measurement is angle θ=Angle of projection(R*V_{ew})−Angle of projection(V_{left}), where R is the Quaternion rotation matrix with rotation given by V_{left}×Vₛₑ and normalized appropriately.

In another example, for the ankle dorsiflexion range of motion measurement (for either left or right), its range of motion measurement has been determined by determining the difference in angle between a down vector V_{down}=(0, −1, 0) and an ankle-to-knee vector Vₐₖ=Pₖ−Pₐ. Pₖ is the skeletal knee position coordinate and Pₐ is the skeletal ankle position coordinate, both on the body side (e.g., left or right) of interest. In other words, ankle dorsiflexion range of motion measurement is angle θ=angle between V_{down} and Vₐₖ.

In another example, for hip rotation, in flexion, its range of motion measurement (for either left or right) has been determined by projecting a three-dimensional knee-to-ankle vector, Vₖₐ=Pₐ−Pₖ, onto a plane perpendicular to a hip-to-knee vector, Vₕₖ=Pₖ to Pₕ and measuring the angle between the projected vector and the projected “left” vector, V_{left}=(1, 0, 0). Pₐ is the three-dimensional skeletal ankle position coordinate, Pₖ is the three-dimensional skeletal knee position coordinate, and Pₕ is the three-dimensional skeletal hip position coordinate, all of the body side (e.g., left or right) of interest. In other words, the hip rotation range of motion measurement is angle θ=Angle of projection(R*Vₖₐ)−Angle of projection(V_{left}), where R is the Quaternion rotation matrix with rotation given by V_{left}×Vₕₖ and normalized appropriately.

In another example, for the hamstring sit and reach range of motion measurement, its range of motion measurement has been determined by measuring the difference in depth/distance between the subject's hands and the subject's feet. For example, the hamstring sit and reach range of motion measurement distance equals the distance between a skeletal foot position coordinate and a skeletal hand position coordinate.

At 504, it is determined whether the received range of motion measurement is within a desired distribution. For example, based on a known statistical (e.g., Gaussian) distribution for the data to be filtered, outlier data is discarded.

In some embodiments, given a set of stored (e.g., stored in circular list, where for each range of motion measurement sample added past a fixed size limit, the earliest sample entry is removed to ensure only the fixed size limit number of range of motion measurements is stored) range of motion measurement samples, S, where each range of measurement sample, s, is a scalar or vector range of motion measurement sample and sεS, the following statistical test is utilized for a received measurement sample s: H₀=erf(|s−μ_{S}|/σ_{S})<T. In this equation, μ_{S} and σ_{S} are mean and standard deviation of the samples of S, respectively, erf( )is the integral of the Gaussian function, and T is the null hypothesis rejection threshold (e.g., 0.99). In some embodiments, if H₀ is true for the received range of motion measurement, the measurement is within the desired distribution and if H₀ is not true for the received range of motion measurement, the measurement is not within the desired distribution.

At 506, the received range of motion measurement is discarded if it is not within the desired distribution. For example, the discarded range of motion measurement is not utilized to determine an output/final range of motion measurement (e.g., not utilized in a group of component samples averaged to determine the output/final range of motion measurement). In another example, the discarded range of motion measurement is not utilized to determine whether a user subject has held a valid pose for at least a threshold amount of time. In some embodiments, if H is the filtered list of one or more samples of S where H₀ is true for the sample, the discarded range of motion measurements is not included in the filtered list of range of motion measurements H. For example, if H₀ is not true for the received range of motion measurement, the measurement is discarded and not included in filtered list H.

At 508, the received range of motion measurement is identified as a valid measurement if it is within the desired distribution. For example, the valid range of motion measurement is utilized to determine an output/final range of motion measurement (e.g., included in a group of component samples averaged to determine the output/final range of motion measurement). In another example, the valid range of motion measurement is utilized to determine whether a user subject has held a valid pose for at least a threshold amount of time. In some embodiments, if H is the filtered list of one or more samples of S where H₀ is true for the sample, the valid range of motion measurements is included in the filtered list of range of motion measurements H. For example, if H₀ is true for the received range of motion measurement, the measurement is discarded and not included in filtered list H.

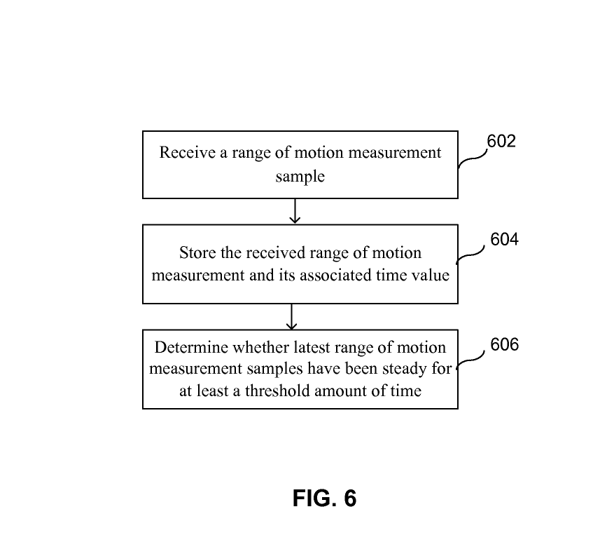

FIG. 6 is a flow chart illustrating an embodiment of a process for determining steadiness of a subject. In some embodiments, at least a portion of the process of FIG. 6 may be performed by camera 102, user device 104 and/or server 106 of FIG. 1. In some embodiments, the process of FIG. 6 is included in 206 of FIG. 2. In some embodiments, the process of FIG. 6 is included in 306 and/or 308 of FIG. 3. In some embodiments, the process of FIG. 6 is included in 410 and/or 412 of FIG. 4. In some embodiments, by detecting steadiness of a user subject, consistent range of motion measurement samples may be measured during the period of steadiness to allow a more reliable and consistent output average range of motion measurement.

At 602, a range of motion measurement sample is received. In some embodiments, the received range of motion measurement sample has been determined using optical data received in 206 of FIG. 2. For example, an updated set of optical data is continually received on a periodic basis and each latest set of optical data is analyzed to determine the range of motion measurement sample for the set. In some embodiments, the range of motion sample is the range of motion measurement sample received in 502 of FIG. 5.

At 604, the received range of motion measurement and its associated time value are stored. In some embodiments, the measurement is only stored if it is determined that the measurement is valid (e.g., filtered). For example, if it is determined that the measurement is valid in 508 of FIG. 5, the received measurement is stored. In some embodiments, the time value is associated with a timestamp of when the pose/optical data processed to determine that the received measurement data was captured. In some embodiments, the time value is associated with an ordering of the received measurement with respect to one or more other range of motion measurement samples.

In some embodiments, the received range of motion measurement sample and the associated time value are stored in a data structure. For example, the received range of motion measurement sample and the associated time value are stored as an entry in a data structure large enough to store all entries corresponding to all optical data captured during a time period (e.g., threshold time period of 412 of FIG. 4). For example, a received measurement, s, and its associated timestamp, t, is stored in a list: (s,t) ε S. The list may be configured to only store latest entries corresponding to a time range, T. In some embodiments, the data structure is a first-in-first-out data structure that only stores data associated with a specified time period. For example, when a new measurement sample (sₖ, tₖ) is received, any entry (s,t) in S where tₖ−t>T is removed from the data structure.

At 606, it is determined whether latest range of motion measurement samples have been steady for at least a threshold amount of time. For example it is determined whether all latest filtered/valid range of motion measurement samples associated with a time value within the last threshold amount of time are within a threshold value range. In some embodiments, the threshold amount of time is the capture threshold amount of time of 412 of FIG. 4. In some embodiments, only latest entries associated with the threshold amount of time can be stored in the data structure of 604. In some embodiments, determining whether the range of motion measurement samples have been steady for at least the threshold amount of time includes determining whether all entries of the data structure storing the measurement in 604 are all within a threshold range. In some embodiments, if all entries of the data structure are within the threshold range, it is determined that the measurement samples have been steady and if not all entries of the data structure are within the threshold range, it is determined that the measurement samples have been not steady.

For example, suppose the group S only stores latest entries corresponding to a threshold time range, T. It is then determined whether max(S)−min(S)<sₜ, where max(S) is the maximum measurement value of entries within S, min(S) is the minimum measurement value of entries within S, and sₜ is the maximum threshold range of variability before measurement data is determined to be not steady. If this equation is true, it is determined that the measurement samples are steady and if this equation is not true, it is determined that the measurement samples are not steady.

In some embodiments, if it is determined that latest range of motion measurement samples have not been steady, the process returns to 602. In some embodiments, if it is determined that latest range of motion measurement samples have been steady, an average of the range of motion measurement samples in the data structure is determined and provided as an output range of motion measurement (e.g., determined as the output range of motion measurement in 206 of FIG. 2 and/or 308 of FIG. 3) and the process of FIG. 6 ends.

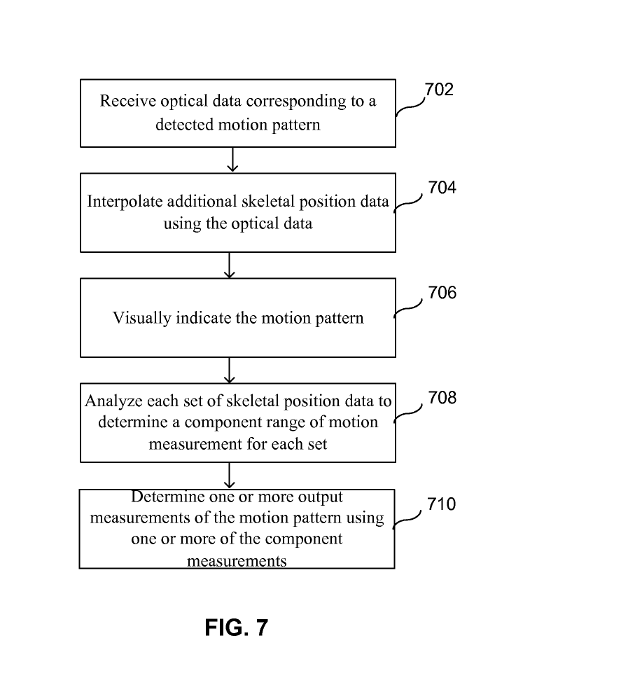

FIG. 7 is a flow chart illustrating an embodiment of a process for capturing a range of motion of a subject for a motion pattern. For example, rather than determining a range of motion measurement for a steady pose of the subject, the range of motion measurement is captured while the subject is performing a gesture/motion/action. An example of the activity includes a baseball pitch using a left/right arm and the range of motion of the appropriate left/right shoulder while performing the baseball pitch in front of a camera is determined. In some embodiments, the process of FIG. 7 is at least in part included in 206 and/or 208 of FIG. 2.

At 702, optical data corresponding to a detected motion pattern is received. In some embodiments, the detected motion pattern corresponds to one of one or more range of motion measurements determined to be captured in 204 of FIG. 2. In some embodiments, data captured by a camera is analyzed to determine whether the motion pattern has been detected. For example, machine learning is utilized to learn to detect a specific motion pattern, and when the learned motion pattern has been detected, an indication is provided along with optical data corresponding to the detected motion pattern.

In some embodiments, the optical data includes data captured using a camera (e.g., camera 102 of FIG. 1). In some embodiments, the optical data includes a plurality of sets of skeletal position data of the subject while the subject performed the detected motion pattern. For example, each set of skeletal position data includes a complete set of skeletal position location coordinates at an instance during the motion pattern. Each different set may capture the subject at different points during the motion pattern. For example, coordinates (2D or 3D) of one or more of the following skeletal positions are provided: right hand, right wrist, right elbow, right shoulder, head, shoulder center, left hand, left wrist, left elbow, left shoulder, spine, hip center, right hip, right knee, right ankle, right foot, left hip, left knee, left ankle, and left foot. In some embodiments, the optical data and identification of the motion pattern are received via a software development kit (i.e., SDK) and/or an application programming interface (i.e., API) of a camera. For example, a 3-D camera (e.g., Microsoft Kinect) captures an image of a subject and processes the image to identify 3-D coordinates of skeletal positions on the body of the subject as well as identify known motion patterns. These 3-D coordinates and identification of the motion pattern may be accessed from the 3-D camera using a SDK/API of the camera (e.g., accessed by user device 104 from camera 102 of FIG. 1).

At 704, additional skeletal position data is interpolated using the optical data. For example, a camera may be only able to capture a limited number of sets of optical data of the motion pattern due to sample rate limitations of the camera. In some embodiments, in order to increase the sample rate and increase the corresponding sets of skeletal position data, additional sets of skeletal position coordinates are interpolated using the known received sets of skeletal position coordinates. For example, a set of position coordinates between two sets of known received sets of skeletal position coordinates is interpolated (e.g., interpolate by averaging two corresponding known skeletal points).

At 706, the motion pattern is visually indicated. For example, the user subject is able to view on a screen the image of the user being captured by a camera optical tracking device. As the subject performs the motion pattern, one or more skeletal points of the user subject may be visually traced and tracked on the screen. For example, a line representing the movement of left wrist positions of the subject is traced on the screen. In some embodiments, the coordinates traced/tracked on the screen may correspond to optical data received in 702 and/or data interpolated in 704. For example, a line connecting the corresponding skeletal points of optical data received in 702 and/or data interpolated in 704 is displayed.

At 708, each set of skeletal position data is analyzed to determine a component range of motion measurement for each set. For example, each set of skeletal position data captures a body position of a subject at a different point in time while performing the motion pattern and the range of motion measurement corresponding to each set is determined. In some embodiments, the range of motion measurement is determined using at least a portion of the process of FIG. 3, which in turn may utilize one or more portions of the processes of FIGS. 4-6. Each set of skeletal position data may include data received in 702 and/or interpolated in 704.

At 710, one or more output measurements of the motion pattern are determined using one or more of the component measurements. For example, a maximum, a minimum, and/or an average range of motion measured of the component range of motion measurements is provided as an output. In some embodiments, other measurements corresponding to the motion pattern may also be provided. For example, a movement speed/velocity (e.g., minimum, maximum, average, etc.) of a specific skeletal point of the subject may be measured and provided as an output. The velocity may be tracked over time for different measurements. In some embodiments, the output measurements are utilized in 208 of FIG. 2 for analysis. For example, the output measurements are compared with previous output measurements to determine an injury risk factor.

FIG. 8 is a diagram illustrating an example embodiment of a user interface visual display of a range of motion capture. Interface 800 (e.g., interface of user device 104 of FIG. 1) shows a greyed image 802 of a subject captured using a camera (e.g., camera 102 of FIG. 1). Visual identification of left shoulder 804, left elbow 806 and left wrist 808 is shown as a visual overlay over image 804 of the subject to identify to a user joint locations of interest for the should rotation measurement being captured. The visual indications of vector 810 that connects left shoulder 804 and left elbow 806 and vector 812 that connects left elbow 806 and left wrist 808 are also shown. The coloring of the visual indications of left shoulder 804, left elbow 806, left wrist 808, vector 810 and vector 812 may be dynamically modified to visually indicate the quality of the measurement captured. For example, the coloring may be dynamically changed to red for incorrect pose, yellow for pose that is close to the acceptable pose, or green for acceptable pose. Progress chart 814 shows a total progress of the range of motion measurements being captured for a current measurement session.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A system for determining a range of motion, comprising:
a processor configured to:
receive tracking information including a plurality of skeletal positions from an optical system;
determine a plurality of vectors between selected ones of the skeletal positions where the plurality of vectors has an angular relationship;
determine whether the angular relationship meets a criteria; and
in the event the angular relationship does not meet the criteria, reject the angular relationship; and
a memory coupled with the processor, wherein the memory is configured to provide the processor with instructions.

2. The system of claim 1, wherein the processor is further configured to determine whether a subject is in a correct pose to determine the range of motion of the subject.

3. The system of claim 2, wherein the processor is further configured to display a visual indication whether the subject is in the correct pose.

4. The system of claim 3, wherein the displaying the visual indication includes selecting a color that corresponds to a measure of correctness of a pose of the subject.

5. The system of claim 2, wherein the processor is further configured to provide an indication of which portion of a detected pose of the subject is incorrect.

6. The system of claim 1, wherein the processor is further configured to determine a second angular relationship of the plurality of vectors that indicates a quality of a pose of a subject in determining the range of motion.

7. The system of claim 6, wherein the determining the second angular relationship includes determining an angle between a first vector that includes a left shoulder skeletal position and a right shoulder skeletal position and a second vector that includes the right shoulder skeletal position and a right elbow skeletal position.

8. The system of claim 1, wherein the processor is further configured to determine a numerical indication of how close a current detected pose of a subject is to an ideal pose for determining the range of motion.

9. The system of claim 1, wherein determining whether the angular relationship meets the criteria includes determining whether the angular relationship is within a desired statistical distribution.

10. The system of claim 9, wherein the desired statistical distribution is associated with a Gaussian distribution of previously determined angular relationship samples.

11. The system of claim 1, wherein rejecting the angular relationship includes not including the angular relationship in a list of angular relationship samples to be utilized to determine the range of motion.

12. The system of claim 1, wherein the processor is further configured to, in the event the angular relationship does meet the criteria, validate the angular relationship.

13. The system of claim 12, wherein validating the angular relationship includes utilizing the is angular relationship as one of a plurality of angular relationship samples that are averaged to determine the range of motion.

14. The system of claim 1, wherein determining whether the angular relationship meets the criteria includes determining whether the angular relationship has been stable at least a threshold amount of time.

15. The system of claim 1, wherein determining whether the angular relationship meets the criteria includes adding the angular relationship and an associated time value to a data structure.

16. The system of claim 15, wherein the data structure is configured to remove any entry with an associated time value that is past a threshold time.

17. The system of claim 15, wherein determining whether the angular relationship meets the criteria includes determining whether a difference between a maximum angular relationship value within the data structure and a minimum angular relationship value within the data structure is below a threshold value.

18. The system of claim 17, wherein rejecting the angular relationship includes determining not to provide an output range of motion value.

19. A method for determining a range of motion, comprising:
receiving tracking information including a plurality of skeletal positions from an optical system;
determining a plurality of vectors between selected ones of the skeletal positions where the plurality of vectors has an angular relationship;
using a processor to determine whether the angular relationship meets a criteria; and
in the event the angular relationship does not meet the criteria, rejecting the angular relationship.

20. A computer program product for determining a range of motion, the computer program product being embodied in a non-transitory computer readable storage medium and comprising computer instructions for:
receiving tracking information including a plurality of skeletal positions from an optical system;
determining a plurality of vectors between selected ones of the skeletal positions where the plurality of vectors has an angular relationship;
determining whether the angular relationship meets a criteria; and
in the event the angular relationship does not meet the criteria, rejecting the angular relationship.
